(19) United States
(12) Patent Application Publication
(10) Pub. No.: US 2024/0148658 A1
(43) Pub. Date: May 9, 2024

(54) ADHESIVE MICROCAPSULES FOR MECHANICALLY-RESPONSIVE THERAPEUTIC DELIVERY

(71) Applicants: THE TRUSTEES OF THE UNIVERSITY OF PENNSYLVANIA, Philadelphia, PA (US); THE UNITED STATES GOVERNMENT AS REPRESENTED BY THE DEPARMENT OF VETERANS AFFAIRS, Washington, DC (US)

(72) Inventors: Daeyeon LEE, Wynnewood, PA (US); Yun Kee JO, Philadelphia, PA (US); Robert Leon MAUCK, Philadelphia, PA (US); George R. DODGE, Philadelphia, PA (US)

(21) Appl. No.: 18/552,711

(22) PCT Filed: Mar. 24, 2022
(86) PCT No.: PCT/US2022/071321

§ 371 Date: Sep. 27, 2023

Related U.S. Application Data
(60) Provisional application No. 63/167,260, filed on Mar. 29, 2021.

(51) Int. Cl. A61K 9/50; A61K 9/00; A61P 29/00

(52) U.S. Cl. CPC A61K 9/5031; A61K 9/0021; A61P 29/00

(57) ABSTRACT

A composition, comprising: a plurality of mechanically-activated microcapsules; a mechanically-activated microcapsule defining a shell and an exterior surface; and the mechanically-activated microcapsule comprising one or more adhesion groups disposed Non the exterior surface of the mechanically-activated microcapsule, the one or more adhesion groups being configured to effect a covalent interaction, a non-covalent interaction, or both between the one or more adhesion groups and a matrix material, the covalent interaction, the non-covalent interaction, or both adhering the mechanically-activated microcapsule to the matrix material. Also provided are related methods and related articles.

### RELATED APPLICATIONS

The present application claims priority to and the benefit of U.S. patent application No. 63/167,260, “Adhesive Microcapsules For Mechanically-Responsive Therapeutic Delivery” (filed Mar. 29, 2021). The entirety of the foregoing application is incorporated herein for any and all purposes.

### GOVERNMENT RIGHTS

This invention was made with government support under AR071340 awarded by National Institutes of Health. The government has certain rights in the invention.

### TECHNICAL FIELD

The present disclosure relates to the field of mechanically-sensitive microcapsules and the field of therapeutic delivery.

### BACKGROUND

Extensive full-thickness wounds, including acute postsurgical incisions, transcutaneous prosthesis, burns, and non-healing ulcers, are susceptible to undesirable bacterial infection due to their hypoxic and protein-rich environments that are ideal for bacterial growth. In particular, the formation of blisters due to various mechanical forces in the wound area, especially over joints that are constantly under motion (e.g., knee, elbow, and finger knuckle), increases the risk of developing severe infection.

Improper treatment of the infection potentially gives rise to the development of biofilms which cause delayed wound healing and sometimes necessitate surgical debridement and in some extreme case amputation.

For clinical wound management, non-woven fabric dressings, such as gauzes, bandages, and cotton wools, are the long-standing used materials and most common options for protecting the wound bed from mechanical trauma, dehydration, and infections. However, the passive release of antibiotics from these conventional fabric-based dressings falls short of providing a reliable and timely treatment of infections in dynamic wound environments. Accordingly, there is a long-felt need in the art for improved wound dressings and therapeutic delivery systems. In particular, systems for programmable treatment of bacterial infections are highly desirable.

### SUMMARY

In meeting the described long-felt needs, the present disclosure provides compositions, comprising: a plurality of mechanically-activated microcapsules; a mechanically-activated microcapsule defining a shell and an exterior surface; and the mechanically-activated microcapsule comprising one or more adhesion groups disposed on the exterior surface of the mechanically-activated microcapsule, the one or more adhesion groups being configured to effect a covalent interaction, a non-covalent interaction, or both between the one or more adhesion groups and a matrix material, the covalent interaction, the non-covalent interaction, or both adhering the mechanically-activated microcapsule to the matrix material.

Also provided are methods, comprising: injecting into a subject (e.g., a human patient or an animal patient) an injectable formulation according to the present disclosure (e.g., any one of Aspects 10-14).

Also disclosed are articles, comprising: a matrix material; and a composition according to the present disclosure (e.g., any one of Aspects 1-9); the composition adhered to the matrix material, and the mechanically-activated microcapsules of the composition being adhered to the matrix by covalent interactions, non-covalent interactions, or both between the one or more adhesion groups of the mechanically-activated microcapsules and the matrix material.

Additionally disclosed are methods, comprising: treating a subject with an article according to the present disclosure (e.g., any one of Aspects 16-24).

### BRIEF DESCRIPTION OF THE DRAWINGS

In the drawings, which are not necessarily drawn to scale, like numerals may describe similar components in different views. Like numerals having different letter suffixes may represent different instances of similar components. The drawings illustrate generally, by way of example, but not by way of limitation, various aspects discussed in the present document. In the drawings:

FIG. 1 illustrates fabrication and characterization of PDA-MAMCs. (A, B) Schematic illustration of (A) the stretch-triggered antibiotics release from fabric wound dressing and (B) the fabrication of the PDA-MAMCs. (C) Microscopic images of a) the emulsification process within a capillary microfluidic device and b) the generated water-in-oil-in-water (W/O/W) double emulsions. (D) Fluorescent images of the MAMCs with labeled shells (red) and PDA-MAMCs with labeled PDA coating layer (green). (E) UV-Vis spectroscopy of the MAMCs, the PDA-MAMCs and a PDA-coated transparent polystyrene surface. Blue regions indicate the characteristic absorbance peaks for dopaminochrome at 388 nm and dimers of dopaminochrome and 5,6-dihyrixindole at 400-450 nm on the PDA-MAMC surface.

FIG. 2 illustrates adhesiveness of PDA-MAMCs. (A) Images showing adhesion of microcapsules to porcine skin and a plastic surface. The right-most images present confocal enlargements of the retained microcapsules on the plastic surface after washing. (B, C) Microcapsule detachment profiles from (B) porcine skin and (C) a plastic surface as a function of centrifugal force (n≥4 specimens, ***p<0.005, **p<0.01, *p<0.05; Kolmogorov-Smirnov test).

FIG. 3 illustrates stretch-induced mechano-activation of PDA-MAMCs in a fibrous matrix. (A) Pseudo-colored SEM images of gauze embedded with MAMCs and PDA-MAMCs. The red spheres indicate the microcapsules. (B) Schematic illustration of the stretch-induced drug release from the adhesive PDA-MAMCs in a fibrous matrix. (C) Schematic illustration showing the stepwise increments in strain as a function of time (left), with strain levels for investigation highlighted in red. Photographs of the PDA-MAMCs-laden gauze before (top) and after (bottom) application of tensile strain (50%). (D) Confocal microscopy images showing changes in the shape of the MAMCs with increasing strain. Fluorescent BSA is released from the aqueous core upon fracture of the MAMCs. (E) Percentage of retained microcapsules (%) and (F) percent of retained microcapsules that are full (%) as a function of tensile strain application. Data represent the mean±standard deviation with statistical significance as indicated (n≥500 microcapsules/loading regimen/type, 4 specimens/loading regimen/type, ***p<0.005; one-way ANOVA test with Tukey's post-hoc test).

FIG. 4 illustrates stretch-responsive antibiotics delivery from the CIF@PDA-MAMCs-laden fabric dressings. (A) A schematic representation of antibacterial assay using groups including no treatment (negative control), intact CIF@PDA-MAMCs, mechano-activated CIF@PDA-MAMCs and free CIF. The CIF@PDA-MAMCs are prepared with CIF in their inner core. Green color indicates presence of CIF in MAMC and in the medium. (B) In vitro release of CIF from the CIF@PDA-MAMCs-laden gauze as a function of tensile strain levels (n≥500 microcapsules/loading regimen/type, 4 specimens/loading regimen/type). (C) Images and (D) area of the inhibition zones formed around the gauzes (n≥3 specimens/type). (E) Viability of E. coli on CIF@PDA-MAMCs-laden gauze after 1 day of culture (n≥3 specimens/type). Data represent the mean±standard deviation with statistical significance as indicated (***p<0.005; one-way ANOVA test with Tukey's post-hoc test).

FIG. 5 provides a size distribution for illustrative MAMCs.

FIG. 6 provides images of MAMCs before and after PDA coating. The brown color becomes darker with the increase of the concentration of PDA.

FIG. 7 illustrates measurement of adhesion strength of PDA-MAMCs. Schematic illustration of (A) the centrifuge method for measurement of adhesion strength and (B) the custom-designed substrate-holding block. β, the angle of the centrifuge tube; h, the height of sample in the centrifuge tube; r, the radial diameter of the samples; rₘₐₓ, the maximum radial diameter of the centrifuge tube.

FIG. 8 illustrates adhesion of PDA-MAMCs on the fabric dressing. (A) Images showing adhesion of MAMCs and PDA-MAMCs on a gauze. (B) Microcapsule detachment profiles from the gauze at different centrifugal forces (n≥4 specimens; ***p<0.005, **p<0.01, *p<0.05; Kolmogorov-Smirnov test).

FIG. 9 illustrates the experimental setup of a custom-built micromechanical tester.

FIG. 10 illustrates stretch-induced mechano-activation of PDA-MAMCs in a fibrous matrix of gauze. SEM images of the gauze loaded with pseudo-red-colored microcapsules before and after stretching.

FIG. 11 provides (A) Thickness of PDA coating layer, PLGA film and PDA-coated PLGA film measured by ellipsometry. (B) Confocal microscopy image of the PDA-MAMCs with labeled PDA coating layer (green). To clearly visualize the PDA layer, non-labeled BSA is used as the aqueous core. (C) Confocal microscopy image of the PDA-MAMCs with labeled PLGA shells (red) used for mechano-activation analyses (% Full>95). To accurately quantify the percentage of full microcapsules, fluorescent BSA (green) is encapsulated into the PDA-MAMC with nonlabelled PDA coating layer.

FIG. 12 provides adhesiveness of PDA-MAMCs at different concentration of PDA coating. (A) Images showing adhesion of microcapsules to porcine skin and a plastic surface. (B, C) Microcapsule detachment profiles from (B) porcine skin and (C) a plastic surface as a function of centrifugal force (n≥4 specimens, ***p<0.005, **p<0.01, *p<0.05; Kolmogorov-Smirnov test).

FIG. 13 provides (A) photographs of a gauze before and after application of tensile strain. (B) Strain-stress curve of the gauze upon uniaxial stretching with 10% stepwise increments at a strain rate of 1%/s.

FIG. 14 provides released CIF from the CIF@PDA-MAMCs-laden gauze immediately (0 h) after or 24 h after rupture induced by stretching with tensile strains of 20% and 50% (n≥500 microcapsules/loading regimen/type, 4 specimens/loading regimen/type).

### DETAILED DESCRIPTION OF ILLUSTRATIVE EMBODIMENTS

The present disclosure may be understood more readily by reference to the following detailed description of desired embodiments and the examples included therein.

Unless otherwise defined, all technical and scientific terms used herein have the same meaning as commonly understood by one of ordinary skill in the art. In case of conflict, the present document, including definitions, will control. Preferred methods and materials are described below, although methods and materials similar or equivalent to those described herein can be used in practice or testing. All publications, patent applications, patents and other references mentioned herein are incorporated by reference in their entirety. The materials, methods, and examples disclosed herein are illustrative only and not intended to be limiting.

The singular forms “a,” “an,” and “the” include plural referents unless the context clearly dictates otherwise.

As used in the specification and in the claims, the term “comprising” may include the embodiments “consisting of” and “consisting essentially of.” The terms “comprise(s),” “include(s),” “having,” “has,” “can,” “contain(s),” and variants thereof, as used herein, are intended to be open-ended transitional phrases, terms, or words that require the presence of the named ingredients/steps and permit the presence of other ingredients/steps. However, such description should be construed as also describing compositions or processes as “consisting of” and “consisting essentially of” the enumerated ingredients/steps, which allows the presence of only the named ingredients/steps, along with any impurities that might result therefrom, and excludes other ingredients/steps.

As used herein, the terms “about” and “at or about” mean that the amount or value in question can be the value designated some other value approximately or about the same. It is generally understood, as used herein, that it is the nominal value indicated ±10% variation unless otherwise indicated or inferred. The term is intended to convey that similar values promote equivalent results or effects recited in the claims. That is, it is understood that amounts, sizes, formulations, parameters, and other quantities and characteristics are not and need not be exact, but can be approximate and/or larger or smaller, as desired, reflecting tolerances, conversion factors, rounding off, measurement error and the like, and other factors known to those of skill in the art. In general, an amount, size, formulation, parameter or other quantity or characteristic is “about” or “approximate” whether or not expressly stated to be such. It is understood that where “about” is used before a quantitative value, the parameter also includes the specific quantitative value itself, unless specifically stated otherwise.

Unless indicated to the contrary, the numerical values should be understood to include numerical values which are the same when reduced to the same number of significant figures and numerical values which differ from the stated value by less than the experimental error of conventional measurement technique of the type described in the present application to determine the value.

All ranges disclosed herein are inclusive of the recited endpoint and independently of the endpoints (e.g., “between 2 grams and 10 grams, and all the intermediate values” includes 2 grams, 10 grams, and all intermediate values). The endpoints of the ranges and any values disclosed herein are not limited to the precise range or value; they are sufficiently imprecise to include values approximating these ranges and/or values. All ranges are combinable.

As used herein, approximating language may be applied to modify any quantitative representation that may vary without resulting in a change in the basic function to which it is related. Accordingly, a value modified by a term or terms, such as “about” and “substantially,” may not be limited to the precise value specified, in some cases. In at least some instances, the approximating language may correspond to the precision of an instrument for measuring the value. The modifier “about” should also be considered as disclosing the range defined by the absolute values of the two endpoints. For example, the expression “from about 2 to about 4” also discloses the range “from 2 to 4.” The term “about” may refer to plus or minus 10% of the indicated number. For example, “about 10%” may indicate a range of 9% to 11%, and “about 1” may mean from 0.9-1.1. Other meanings of “about” may be apparent from the context, such as rounding off, so, for example “about 1” may also mean from 0.5 to 1.4. Further, the term “comprising” should be understood as having its open-ended meaning of “including,” but the term also includes the closed meaning of the term “consisting.” For example, a composition that comprises components A and B may be a composition that includes A, B, and other components, but may also be a composition made of A and B only. Any documents cited herein are incorporated by reference in their entireties for any and all purposes.

Microcapsules are capable of protecting encapsulated therapeutics within a solid shell against degradation and environmental factors and controlling their release based on the characteristics of the shell. Various types of stimuli-responsive microcapsules that can release their contents in response to heat, chemical, and light activation have been successfully demonstrated.

One endogenous stimulus that has not been taken advantage of to enable self-activation of drug release from microcapsules, despite their importance of mechanical forces in human physiology, is the mechanical deformation and mechanical loading that accompanies a variety of body motions and functions. To realize effective strain-triggered release of therapeutics from mechanically activated microcapsules (MAMCs) within fibrous matrices such as those used in wound dressings, MAMCs need to have strong interactions with the fibrous substrate of the dressing, especially in a physiologically hydrated condition. Conventional methods for binding microcapsules onto fabrics, however, often result in considerable toxicity, low adhesion, and binder-induced film formation which could hinder the release of encapsulated drugs.

### EXEMPLARY DISCLOSURE

The following disclosure is illustrative only and does not limit the scope of the present disclosure or the appended claims.

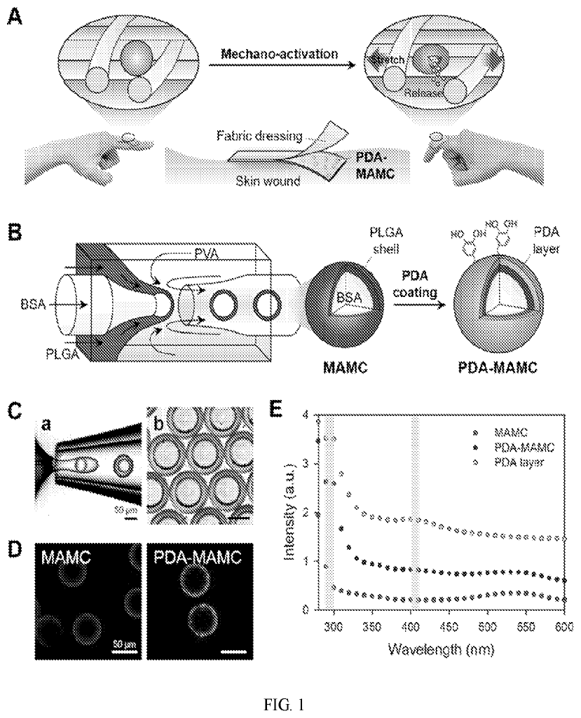

As a non-limiting illustration of the disclosed technology, we provide a mechano-responsive (e.g., stretch-sensitive) delivery system by imparting a relatively strong adhesion between MAMCs and a fibrous matrix via a mussel-inspired coating to enable mechanically activated release of antibiotics from non-woven fabric dressings in response to tensile strains. Polydopamine (PDA) is one non-limiting strategy to functionalize the surface of a wide variety of materials. The hydrated adhesive properties as well as biocompatibility and biodegradability make PDA particularly suitable for biomedical applications. We hypothesize that PDA coating will markedly enhance the adhesion of MAMCs onto fibrous substrates in hydrated environments, and that the release of antibiotics from the adhesive MAMCs can be triggered by stretching of the dressing (FIG. 1A).

We prepare a uniform population of the PDA-coated MAMCs (PDA-MAMCs) using a microfluidic technique followed by oxidative dopamine polymerization (FIG. 1), and evaluate their adhesive properties and stretch-responsive mechano-activation functionality. The tensile strain-dependent release profile and antibacterial performance of ciprofloxacin (CIF)-loaded PDA-MAMCs embedded in an ordinary gauze are also assessed.

Results and Discussion

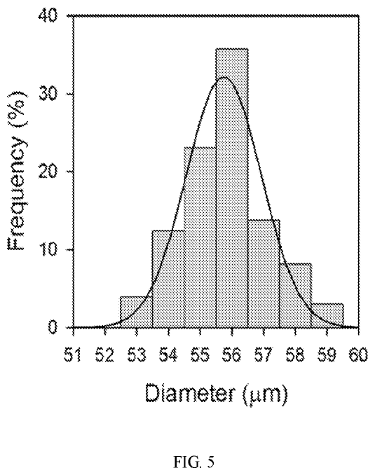

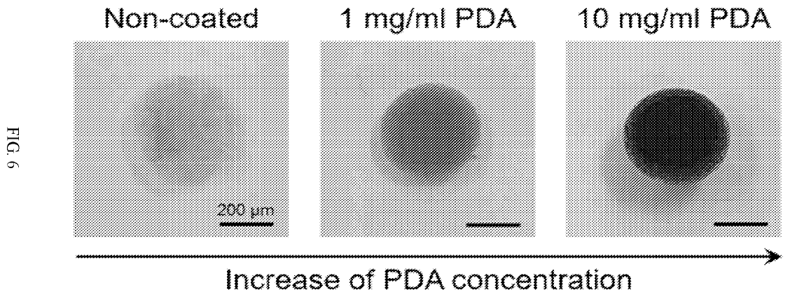

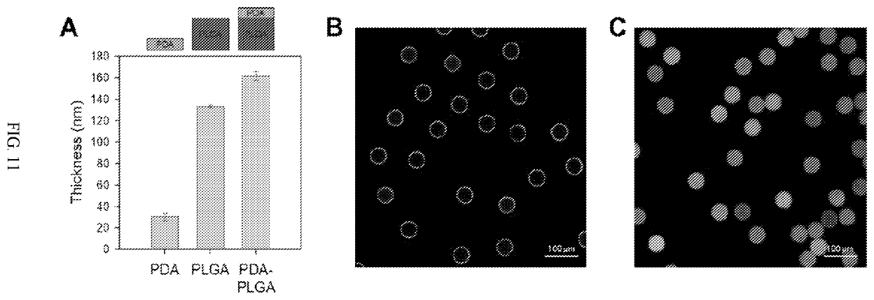

Monodisperse poly(D,L-lactide-co-glycolide) (PLGA)-based MAMCs with a diameter of ˜56 μm and a shell thickness of ˜0.95 μm are fabricated by generating water-in-oil-in-water (W/O/W) double emulsions using a glass capillary microfluidic device, followed by solvent removal (FIG. 1C and FIG. 5). Nile Red is added to the middle phase to fluorescently label the shell of MAMCs, facilitating their visualization. Incubation of the resulting MAMCs in an alkaline dopamine solution (pH 8.5) results in the formation of PDA coating on the MAMC surface, as determined by fluorescence imaging (FIG. 1D). The successful coating is also confirmed by a distinct color change of the PDA-MAMC surface to light brown due to the oxidation of the dopamine monomers (FIG. 6). Consistent with this result from UV-Vis spectroscopy, we observe the characteristic absorbance peaks for dopaminochrome at 388 nm and dimers of dopaminochrome and 5,6-dihyrxyindole at 400-450 nm on the PDA-MAMC surface (FIG. 1E). From the ellipsometry result, the thickness of PDA-coated PLGA film is equivalent to the sum of thicknesses of PDA coating layer and PLGA film (FIG. 11A), suggesting that PDA likely is not infiltrating the PLGA film. Similarly, the PDA coating layer is located along the outer outline of PLGA shell without penetrating into the core of MAMC, which is confirmed by confocal microscopy (FIG. 11B).

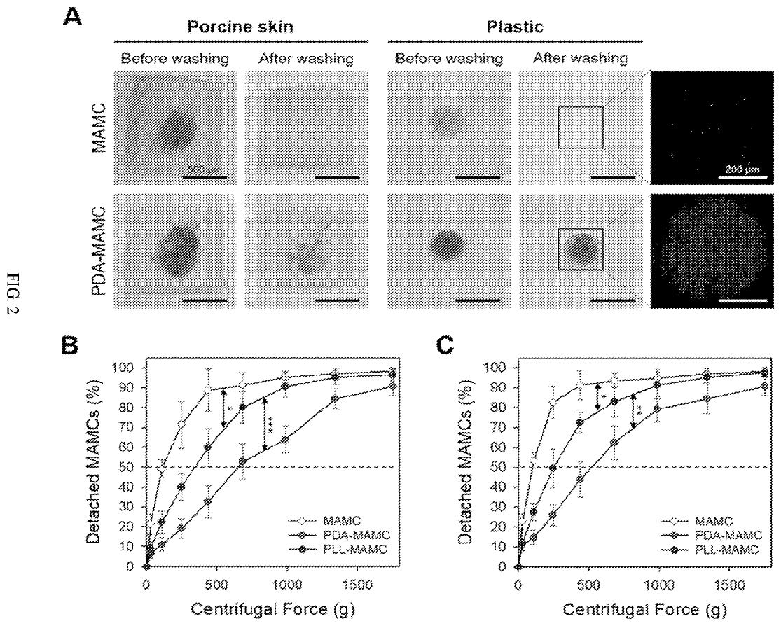

The adhesive property of the PDA-MAMCs is evaluated by testing their ability to adhere to either the surface of porcine skin or a plastic surface under a hydrated condition (FIG. 2A). As-prepared MAMCs without PDA coating in Tris-HCl buffer are used as a negative control. The majority of the PDA-MAMCs remain adhered on both surfaces after rinsing with distilled water (DW), whereas the majority of the unmodified MAMCs are washed away from the surface, indicating superior adhesiveness of the PDA-MAMCs on wet substrates independent of the nature of the surface.

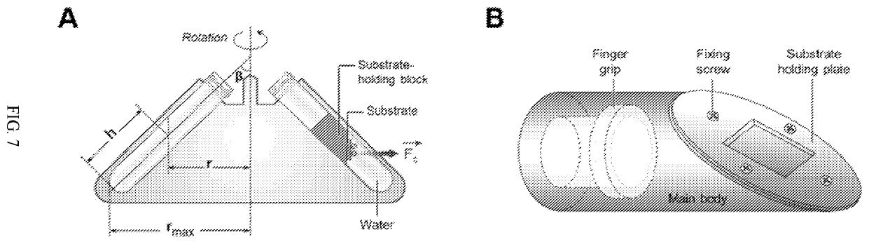

The adhesion strength of the PDA-MAMC is quantitatively characterized by a centrifugation approach. In this method, a custom-designed substrate-holding block is used to maintain the substrate parallel to the centrifugal axis, allowing microcapsules on the substrate to experience only the centrifugal force (FIG. 7). Cationic poly-L-lysine (PLL) is coated onto the MAMC surface (PLL-MAMC) via simple agitation of the MAMCs in a PLL solution to serve as a positive control and to compare the adhesion strength to that of PDA-MAMCs.

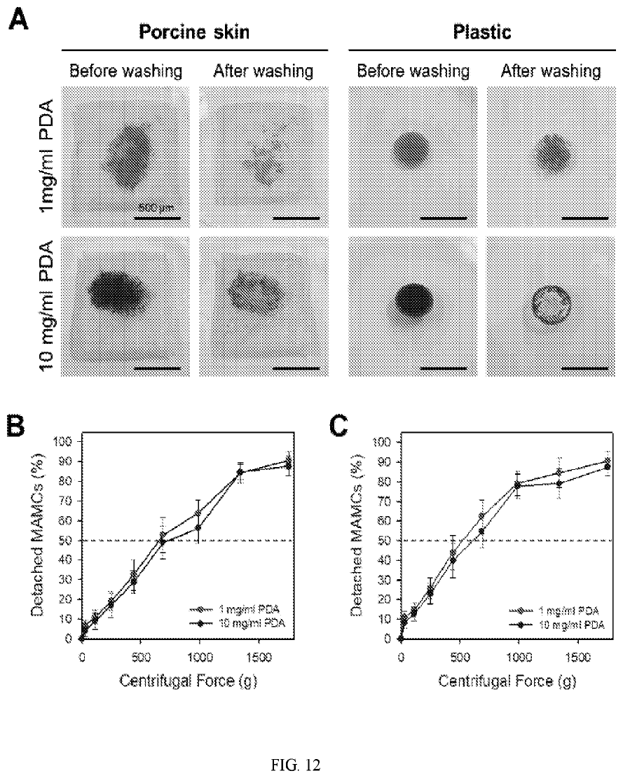

The fraction of MAMCs that are removed from the surfaces of the two substrates as a function of centrifugal force clearly show that the PDA-MAMC has the strongest adhesion to both the porcine skin and the plastic surfaces. The adhesion force of PDA-MAMCs onto the plastic surface is 5 times higher compared to unmodified MAMCs and 2 times higher than PLL-MAMCs (Table 1 and FIG. 2B,C). All three types of MAMCs adhere more avidly to the porcine skin than to the plastic surface, which is likely due to the relatively higher surface roughness of the porcine skin. Again, PDA-MAMCs show the greatest increase in the adhesion strength on the porcine skin; the enhanced adhesion of PDA-MAMCs on the porcine skin could be due to the formation of covalent bonds between the catechol groups and the amine or thiol functional groups on the surface of porcine skin via Michael addition or Schiff base reactions. An increase in the concentration of PDA coating (from 1 mg/ml to 10 mg/ml) on MAMCs does not result in an increase in adhesion strength on both the porcine skin and the plastic surfaces (FIG. 12), probably due to the full coverage of PDA coating layer on the surface of MAMCs (FIG. 11) and the saturation of adhesion strength when coated with 1 mg/ml PDA.

TABLE 1
Adhesion strength of PDA-MAMCs to porcine
skin, a plastic surface and gauze (n ≥ 4).

| Substrate | MAMC | PDA-MAMC | PLL-MAMC |
|---|---|---|---|
| Porcine skin | 112 ± 29 g | 633 ± 106 g | 321 ± 77 g |
| Plastic | 94 ± 19 g | 510 ± 77 g | 243 ± 58 g |
| Gauze | 372 ± 63 g | 1,092 ± 94 g | 610 ± 104 g |

TABLE 2
Adhesion strength of PDA-MAMCs at different concentration of
PDA coating to porcine skin and a plastic surface (n ≥ 4).

| Substrate | 1 mg/ml PDA | 10 mg/ml PDA |
|---|---|---|
| Porcine skin | 633 ± 106 g | 689 ± 93 g |
| Plastic | 510 ± 77 g | 608 ± 109 g |

All three types of MAMCs adhere more avidly to the porcine skin than to the plastic surface, which is likely due to the relatively higher surface roughness of the porcine skin. PDA-MAMCs show the greatest increase in the adhesion strength on the porcine skin; the enhanced adhesion of PDA-MAMCs on the porcine skin could be due to covalent bonds between the catechol groups and the amine or thiol functional groups on the surface of porcine skin via Michael addition or Schiff base reactions.

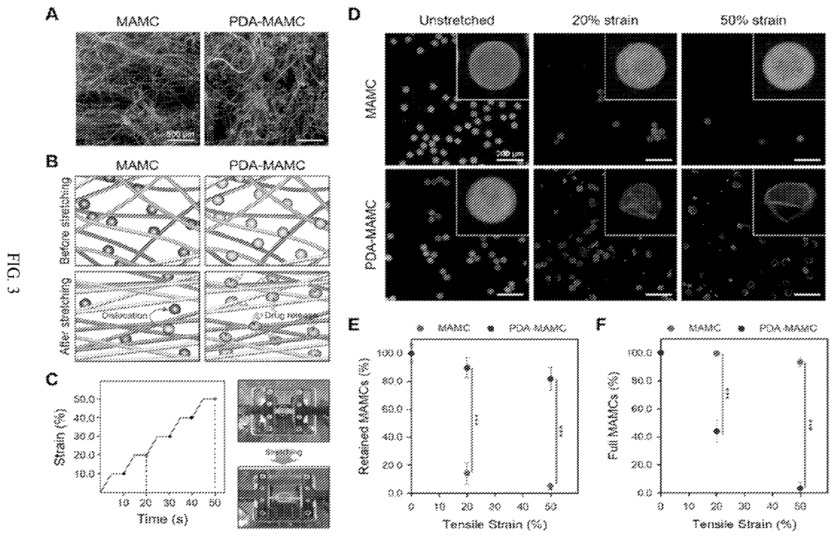

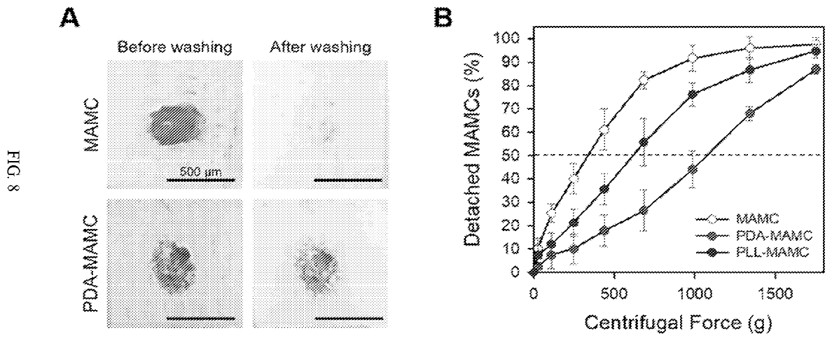

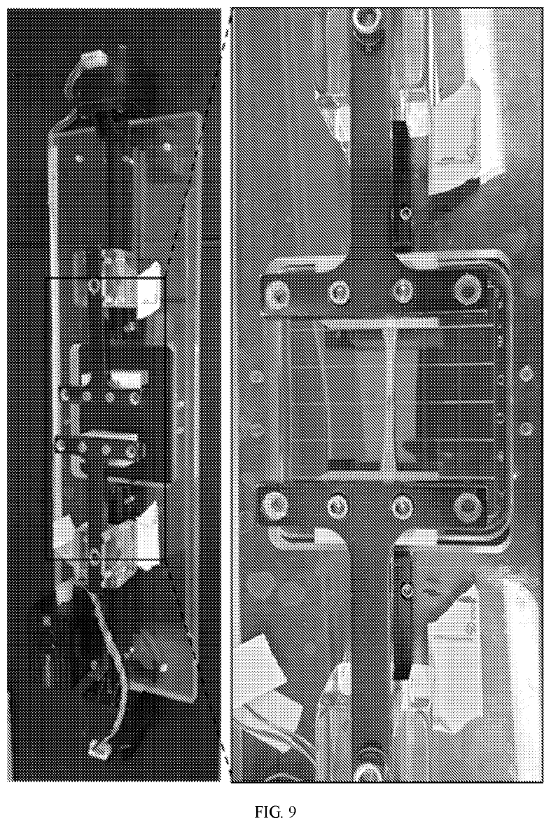

To test PDA-MAMCs as strain-responsive drug carriers for wound dressing, we assess their release properties in a three-dimensional (3D) fibrous matrix of a commercial non-woven gauze under physiologically relevant strains. MAMCs are loaded into the gauze by placing a drop of MAMC suspension on top of the gauze and subsequently rinsing with DW. PDA-MAMCs exhibit a higher level of retention in the stretchable fabric gauze under a wet condition, compared to the unmodified MAMCs, suggesting the strong adhesiveness of the PDA-MAMC to the fibrous matrix (FIG. 3A and FIG. 8). One can (without being bound to any particular theory) hypothesize that PDA-MAMCs that have comparatively strong adhesion to the fibers will experience compression and/or shear forces during stretching of the gauze, resulting in the mechano-activation and rupture of the PDA-MAMCs (FIG. 3B). To test this hypothesis, a PDA-MAMCs-laden gauze is subjected to stepwise grip-to-grip strains in uniaxial tension using a custom-designed micromechanical device (FIG. 3C and FIG. 9). The shell and core of MAMCs are loaded with Nile Red and fluorescently labeled bovine serum albumin (BSA), respectively, to facilitate the characterization of mechano-activation and release of the cargo. MAMCs are used after confirming that the percentage of full microcapsules (% Full) is over 95 (FIG. 11C).

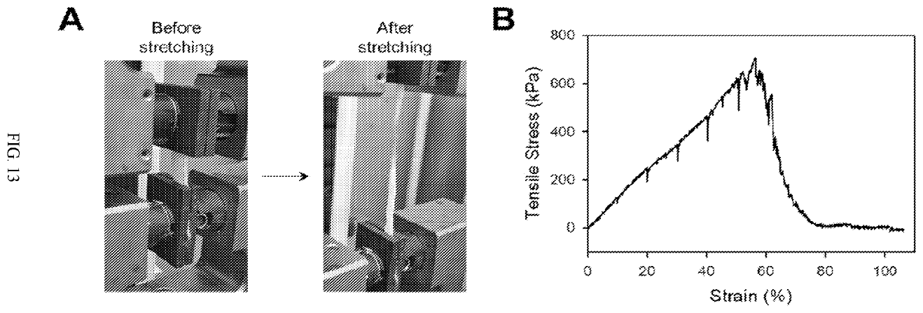

When 20% tensile strain is applied, ˜85% of the unmodified MAMCs detach from the fibrous network, while the majority of the PDA-MAMCs remain adhered on the gauze fibers (FIG. 3D,E). Upon stretching to 50% strain, approximately 80% of the PDA-MAMCs remain within the gauze due to their strong adhesion to the fibers, whereas very few unmodified MAMCs remain under the same conditions. Since the gauze shows a plastic deformation and a breakage when tensile strain exceeds 50% (FIG. 13), strains of up to 50% are applied to the PDA-MAMCs-laden gauze. More than half of the remaining PDA-MAMCs exhibit clear deformations after 20% strain of the gauze, with many losing their structural integrity. Loss of structural integrity is evidenced by the loss of fluorescent signal from the core of the PDA-MAMCs in ˜97% of microcapsules, as shown in the 3D volume reconstruction of confocal z-stack images, indicating the release of their content (BSA) (FIG. 3D,F). The application of 50% tensile strain, physiologically relevant in the bending of knees, finger joints and wrists, results in the mechano-activation of almost all of the PDA-MAMCs within the fibrous network.

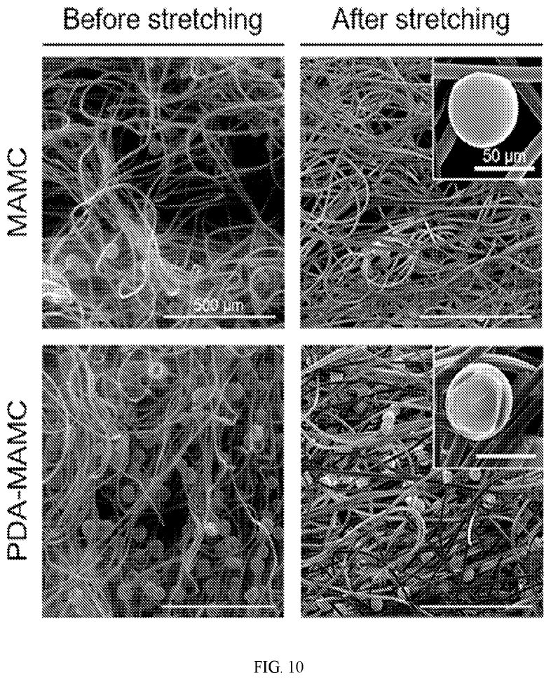

The shape of ruptured MAMCs show anisotropy, indicating that they experience a compression induced by the geometric change of the fibrous network upon uniaxial stretching (FIG. 10). In contrast, most of the unmodified MAMCs retain their spherical shape without detectable release of encapsulated fluorescent BSA, even after stretching the gauze to 50% strain. These unmodified MAMCs are likely dislodged from the fiber substrate during the gauze deformation, and so mechanical force is not transmitted to them. Collectively, these findings suggest that the adhesive interaction between the PDA-MAMCs and the underlying fiber substrate is essential for an effective mechano-activation of the PDA-MAMCs with uniaxial stretching of the gauze and that this enables a self-regulated release of the therapeutic cargos in a highly spatiotemporally controlled manner.

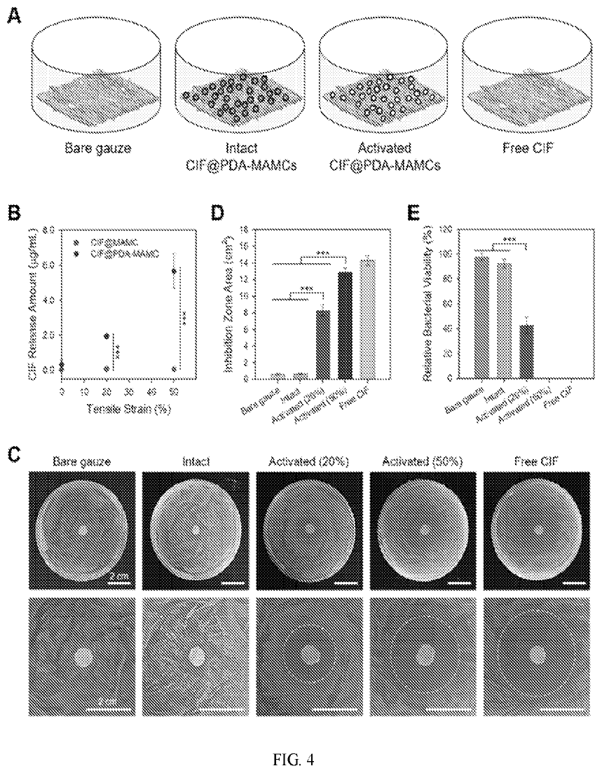

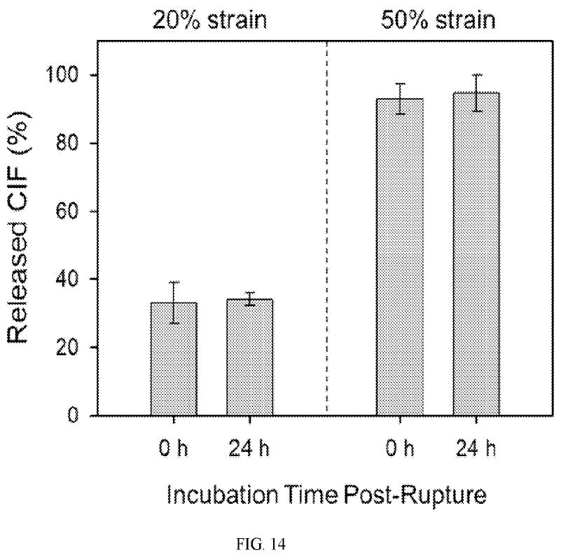

To evaluate PDA-MAMCs to promote the release of antibiotics from fibrous matrices in response to a tensile strain, PDA-MAMCs containing CIF are prepared and loaded into the gauze. To support their utilization in this manner, we chose the potent gram-negative antibiotic CIF that is used clinically to treat a number of bacterial infections, including bone, joint, and skin infections. The CIF-loaded PDA-MAMCs (CIF@PDA-MAMCs) are mechano-activated by stretching the gauze to 20% and 50% strains and the solutions containing released CIF are collected (FIG. 4A). A plain unaltered gauze (a negative control), an intact CIF@PDA-MAMCs-laden gauze (a non-activated control), and a gauze treated with the equivalent amount of free CIF (a positive control) are used for comparison. Similar to the results from confocal microscopy, stretching of the gauze induces the release of CIF from the CIF@PDA-MAMCs, with an increasing amount of CIF released with strain; conversely, CIF-loaded unmodified MAMCs (CIF@MAMCs) do not show any significant release of CIF with stretching (p<0.05, FIG. 4B). In addition, the amounts of released CIF from the CIF@PDA-MAMCs measured immediately after and at 24 h after stretching are not different, which suggests a burst release of CIF from the PDA-MAMCs upon rupture (FIG. 14).

The antibacterial performance of the mechano-activated CIF@PDA-MAMCs is assessed by the disk diffusion test using Escherichia coli (FIG. 4C). Intact CIF@PDA-MAMCs without mechanical activation exhibit negligible inhibition of bacterial growth, indicating that the CIF remains encapsulated in the PDA-MAMCs during the period of incubation. In contrast, significant inhibition zones are observed around CIF@PDA-MAMCs-laden gauze activated by 20% strain of stretching. The diameter of inhibition zones increases with an increase in the strain of mechano-activation, indicating a larger amount of CIF released from the CIF@PDA-MAMCs (FIG. 4D). The mechano-activation of the CIF@PDA-MAMCs at 50% strain achieves an inhibitory zone with a diameter comparable with that of administration of free CIF. The viability of E. coli on CIF@PDA-MAMCs-laden gauze in liquid media is also investigated after 1 day of incubation (FIG. 4E). Mechano-activated CIF@PDA-MAMCs-laden gauze significantly reduces the viability of E. coli at the end of the incubation period compared to non-activated CIF@PDA-MAMCs-laden gauze, consistent with the disk diffusion results. The efficacy of CIF@PDA-MAMCs-laden gauze on the inhibition of E. coli growth also increases with an increase in applied strain. Notably, incubation of bacterial cells on CIF@PDA-MAMCs-laden gauze that was stretched to 50% strain leads to more than 95% loss of bacterial viability. These results clearly demonstrate that stretching with different strain levels enables self-regulated release of antibiotics. Such an approach can be readily extended to various therapeutic agents such as pain relievers and anti-inflammatory agents or growth factors to promote tissue repair involving dermal and wound healing applications.

In conclusion, we developed stretch-responsive therapeutic MAMCs to achieve self-regulated release of an antibiotic from fabric wound dressings. Inspired by the adhesion capability of bivalve mollusks and exploiting a novel microfluidic encapsulation technique, we achieve strong adhesive interactions between drug-delivering PDA-MAMCs and the surrounding fibrous matrix of gauze under hydrated conditions, as well as robust mechano-activation of the PDA-MAMCs in response to uniaxial stretching of the gauze.

Our findings highlight that the strain-dependent release of bioactive contents from PDA-MAMCs can enable a versatile approach to modulate the administration timing of therapeutics in response to body motions, such as bending of joints or exogenously applied pressure. The use of PDA-MAMCs for controlled release of antibiotics to inhibit bacterial growth is validated, providing avenues for programmable antibacterial performance of fabric wound dressings. PDA-MAMCs can thus be successfully employed to prevent undesired bacterial infections in the treatment of wounds, injuries, or surgical sites, ranging from skin to internal tissues with notable strains are inherent or applied. The clinical applications and variety of therapeutic molecules that can be delivered using the disclosed MAMC delivery platform are far-reaching.

Methods

Preparation of PDA-MAMCs

The MAMCs are fabricated using a glass capillary microfluidic device to generate W/O/W double emulsions as previously described. An aqueous solution of 1 mg/mL BSA (Sigma-Aldrich, St Louis, MO, USA) as a model drug or 5% (w/v) of the antibiotic CIF (Sigma-Aldrich) for antibacterial activity analyses is loaded in the inner core of the MAMCs. The middle phase consists of 85:15 PLGA (0.55-0.75 dL/g, ester-terminated; Lactel, Birmingham, AL, USA) dissolved in chloroform with the addition of 100 μg/mL Nile Red (Sigma-Aldrich) to fluorescently label the shell. The outer aqueous phase contains 2% (w/v) poly(vinyl alcohol) (PVA; Sigma-Aldrich). The generated double emulsions are left in the collecting solution of 0.1% (w/v) BSA in phosphate buffered saline (PBS, pH 12; Sigma-Aldrich) for 72 h to allow evaporation of chloroform from the middle phase and hardening of the shell wall.

After solidification, MAMCs are collected and washed with 10 mM Tris-HCl (pH 8.5; Sigma-Aldrich) solution. Subsequently, MAMCs are coated with PDA by immersing them in 10 mM Tris-HCl solution containing 1 mg/ml dopamine-hydrochloride (Sigma-Aldrich) with constant shaking at 100 rpm under ambient conditions for 12 h. As a positive control for the adhesion study, MAMCs are alternatively treated with 1 mg/ml PLL (Sigma-Aldrich) dissolved in PBS.

For visualization of the PDA layer, fluorescein isothiocyanate (FITC) isomer 1 (Sigma-Aldrich) is added to the reacting solution. After rinsing with fresh Tris-HCl solution three times to remove residual dopamine monomers, PDA-MAMCs are collected in Tris-HCl and transferred to PBS (pH 7.4) just prior to use. The prepared PDA-MAMCs are observed by a confocal microscope (20× magnification, mid-plane imaging; Fluoview FV 1000; Olympus, Shinjuku, Tokyo, Japan) and the average outer diameter is measured using the ImageJ software (v.1.52; National Institutes of Health, Bethesda, MD, USA). The presence of PDA coating is also confirmed by monitoring the color change of MAMC suspensions and measuring the absorbance with a comparison with a PDA-coated transparent polystyrene surface using an UV-Vis spectrometer (Infinite M200; TECAN, Zurich, Switzerland). The concentration of PDA-MAMCs is defined as the number of microcapsules/ml (n≥3 aliquots per fabrication batch) as measured under an optical microscope (Eclipse TE200; Nikon, Minato, Tokyo, Japan) within the first 3 days.

Adhesion Analyses

To characterize the adhesion properties of PDA-MAMCs on a biologically relevant matrix, a porcine skin sheet (Stellen Medical, Saint Paul, MN, USA), a plastic surface (Thermo Fisher Scientific, Waltham, MA, USA) and a gauze pad (CVS Pharmacy, Inc., Woonsocket, RI, USA) are used as a model fabric wound dressing. For a qualitative analysis, ˜1,000 microcapsules are placed onto the substrates under hydrated conditions and washed with DW after 30 min. The adhesion strength of the microcapsules is quantitatively measured using a custom centrifugal method that we adopted. A polyurethane-based substrate-holding block is custom-designed and 3D-printed to maintain the substrate parallel to the centrifugal axis during centrifugation (FIG. 7). Microcapsules are placed onto the substrates (10 mm×10 mm) and left for 30 min to allow for surface adhesion. The microcapsules-adhered substrate is firmly fixed to the holding block and loaded into a centrifuge tube, which is filled with deionized water. After centrifugation, adhesion strength (as defined by the force necessary to detach one half of the microcapsules, n≥4 specimens) is measured by counting the number of remaining microcapsules using a digital microscope (5-MP; Celestron, Torrance, CA, USA) and converting the centrifugal force to a normal force according to the equipment manual of the centrifuge (Allegra X-12; Beckman coulter, Brea, CA, USA).

Stretch-Responsive Mechano-Activation Analyses

To monitor mechano-activation in a 3D fibrous matrix, the microcapsules are embedded in a commercial grade gauze (a piece of 10 mm×50 mm; CVS Pharmacy) in the same way they were adhered onto the model surfaces. The morphological analysis of microcapsules-laden gauzes is performed using a scanning electron microscope (SEM; Quanta 600 FEG ESEM; FEI, Hillsboro, OR, USA). For uniaxial stretching of the microcapsules-laden gauze, a custom micromechanical test device is used. Samples are kept hydrated in PBS throughout testing. Then, grip-to-grip tensile strains of 20% or 50% are applied in 10% stepwise increments at a strain rate of 1%/s (FIG. 2C). After application of the tensile deformation, the detached microcapsules are collected and counted to determine the percentage of microcapsules retained in the gauze (n≥500 microcapsules/loading regimen/type, 4 specimens/loading regimen/type). The stretched gauzes are incubated in PBS overnight under ambient condition to allow complete diffusion of inner contents post-rupture. The percentage of full (intact) microcapsules after load are measured using the maximum intensity projections obtained from the confocal z-stacks (10× magnification, n≥500 microcapsules/loading regimen/type, 4 specimens/loading regimen/type).

Antibiotic Delivery and Analyses

To evaluate efficacy of delivering antibiotics in response to tensile strain-induced mechano-activation, 5% (w/v) CIF is encapsulated in inner core of the microcapsules. After stretching to strains of 20% or 50%, 50 μL of fresh PBS solution is dropped onto the microcapsules-laden gauze to collect the released CIF. The released CIF is quantified by measuring the absorbance at 277 nm using the UV-Vis spectrometer (n≥500 microcapsules/loading regimen/type, 4 specimens/loading regimen/type).

The impact of CIF delivery from mechano-activated PDA-MAMCs is also assessed by analyzing antibacterial performances of the microcapsule-loaded gauzes against E. coli JM109 strain using the Kirby-Bauer method. Gauze specimens of 8 mm diameter are placed on Luria-Bertani (LB) agar plates (Sigma-Aldrich) seeded with 100 L of log-phase bacterial cells, and are incubated at 37° C. for 24 h. The area of inhibition zone is measured using Image J (n≥3 specimens/type). The colony forming units (CFU) are also counted after incubation of the microcapsule-loaded gauzes at 37° C. for 24 h in 500 μL of LB medium seeded with bacterial cells in a log-phase at 1:100 (v/v) in a tissue culture plate. Relative bacterial cell viability is determined by dividing the CFU count in culture broth with the gauze specimens or soluble CIF by the CFU count without the gauze (n≥3 specimens/type).

Statistical Analyses

All data are obtained from independent experiments carried out at least in triplicate (n≥3). After Shapiro-Wilk test to evaluate the normality of data distribution, the significance of the data obtained from each group is statistically analyzed via one-way ANOVA test with Tukey's post-hoc test (for a normal distribution) or Kruskal-Wallis test with Dunn's post-hoc test (for a non-normal distribution). Cumulative distribution plots for the detachment of microcapsules in centrifuge-based adhesion analyses are analyzed via Kolmogorov-Smirnov test. The data represent the mean±standard deviation with statistical significance as indicated (*p<0.05, **p<0.01, and ***p<0.005). All data are processed using the R software (v.3.2.1; R Development Core Team, Vienna, Austria).

Aspects

The following Aspects are illustrative only and do not limit the scope of the present disclosure or the appended claims.

Aspect 1. A composition, comprising: a plurality of mechanically-activated microcapsules; a mechanically-activated microcapsule defining a shell and an exterior surface; and the mechanically-activated microcapsule comprising one or more adhesion groups disposed on the exterior surface of the mechanically-activated microcapsule, the one or more adhesion groups being configured to effect a covalent interaction, a non-covalent interaction, or both between the one or more adhesion groups and a matrix material, the covalent interaction, the non-covalent interaction, or both adhering the mechanically-activated microcapsule to the matrix material.

A matrix material can be, e.g., a woven material, a non-woven material, a porous material, a non-porous material, and the like. Fabrics are considered suitable matrix materials, including fabrics used in wound dressings and other healthcare applications. A matrix material can be fibrous in nature, but this is not a requirement, as a matrix material can be non-fibrous, e.g., a porous polymer ribbon or strip. Example matrix materials include, e.g., collagen, cotton, silk, hemp, cellulose, alginates, hydrogels, hydrocolloids, polymers (e.g., polyurethane, nylon, polyester), and the like.

Aspect 2. The composition of Aspect 1, wherein the one or more adhesion groups comprise a 1,2-dihydroxybenzene group. Polydopamine and gallic acid (or a derivative thereof) are considered suitable; phenolic acids that comprise two, three, or more hydroxyl groups are considered particularly suitable.

Aspect 3. The composition of any one of Aspects 1-2, wherein the mechanically-activated microcapsule is characterized as biodegradable.

Aspect 4. The composition of any one of Aspects 1-3, wherein the mechanically-activated microcapsule comprises poly(D,L-lactide-co-glycolide).

Aspect 5. The composition of any one of Aspects 1-4, wherein the mechanically-activated microcapsule comprises a material enclosed within the shell of the mechanically-activated microcapsule.

Aspect 6. The composition of Aspect 5, wherein the material comprises a therapeutic, an analgesic, anti-inflammatory, an antibiotic, or any combination thereof.

Aspect 7. The composition of Aspect 6, wherein the material comprises an antibiotic.

Aspect 8. The composition of any one of Aspects 1-7, wherein the shell defines a thickness in the range of from about 0.05 μm to about 30 μm, e.g., from about 0.05 μm to about 30 μm, from about 0.1 μm to about 25 μm, from about 0.5 μm to about μm, from about 1 μm to about 15 μm, or even from about 3 μm to about 10 μm.

Aspect 9. The composition of any one of Aspects 1-8, wherein a mechanically-activated microcapsule defines a diameter of from about 0.5 μm to about 300 μm, e.g., from about 0.5 μm to about 300 μm, from about 1 μm to about 275 am, from about 5 μm to about 250 am, from about 10 μm to about 225 am, from about 20 μm to about 200 am, from about 30 μm to about 180 am, from about 40 μm to about 150 am, from about 75 μm to about 120 am, or from about 90 μm to about 110 am.

Aspect 10. An injectable formulation, comprising: a composition according to any one of Aspects 1-9; and a carrier, the injectable formulation being configured for injection to a subject.

Aspect 11. The injectable formulation of Aspect 10, wherein the matrix is a selected tissue of the subject.

Aspect 12. The injectable formulation of Aspect 11, wherein the selected tissue is characterized as being in a disease state.

Aspect 13. The injectable formulation of Aspect 12, wherein the disease state is a state of inflammation.

Aspect 14. The injectable formulation of any one of Aspects 11-13, wherein the one or more adhesion groups are configured to adhere preferentially to the selected tissue.

Aspect 15. A method, comprising: injecting into a subject an injectable formulation according to any one of Aspects 10-14.

Aspect 16. An article, comprising: a matrix material; and a composition according to any one of Aspects 1-9; the composition adhered to the matrix material, and the mechanically-activated microcapsules of the composition being adhered to the matrix by covalent interactions, non-covalent interactions, or both between the one or more adhesion groups of the mechanically-activated microcapsules and the matrix material.

Adhesion can be via covalent bonds, ionic bonds, pi-pi stacking, molecular bonding (e.g., hydrogen bonding), or any combination thereof.

The mechanically-activated microcapsules (MAMCs) can exhibit an adhesion strength to the matrix (as measured herein) of from about 100 to 1200 g, from about 200 to about 1200 g, from about 300 to about 1200 g, from about 400 to about 1200 g, from about 500 to about 1200 g, from about 600 to about 1200 g, from about 700 to about 1200 g, from about 800 to about 1200 g, from about 900 to about 1200 g, from about 1000 to about 1200 g, or from about 1100 to about 1200 g. The adhesion strength can be from about 200 to about 1200 g, from about 300 to about 1100 g, from about 400 to about 1000 g, from about 500 to about 900 g, from about 600 to about 800 g, or even about 700 g, including all intermediate ranges, values, and sub-ranges. For example, the adhesion strength can be between any values between about 100 and about 1200 g, in about 50 g increments, e.g., from about 300 g to about 750 g, from about 350 g, to about 700 g, from about 400 g to about 650 g, from about 450 g to about 600 g, or even from about 500 to about 550 g.

An article can be, e.g., configured as a wound dressing. The article can include an adhesive region, which adhesive region can be configured to adhere the article to a user, e.g., to a wound. (A peelable layer can be present to protect the adhesive until the time of use.) An article can be configured to be otherwise attached to a user, e.g., by suture, stapling, and the like. An article can be configured for external application, e.g., application to a user's skin. Such an article can include one or more additional layers, which layers can include, e.g., a hydrophobic layer, a hydrophilic layer, and the like.

An article can also be configured for internal administration to a user, e.g., to be sutured in place within a user, e.g., to a user's muscle or joint. Such an article can include wings or other features (such as apertures) that are configured to receive sutures, staples, or other attachments that secure the article to the user.

Aspect 17. The article of Aspect 16, wherein the matrix material is a fibrous material.

Aspect 18. The article of Aspect 16, wherein the matrix material is a hydrogel. A hydrogel can be, e.g., a pH-sensitive hydrogel. Such hydrogels include, e.g., poly(acrylic acid) and N,N 9-diethylaminoethyl methacrylate; others include poly(acrylamide) (PAAm), poly(methacrylic acid) (PMAA), poly(diethylaminoethyl methacrylate) (PDEAEMA), and poly(dimethylaminoethyl methacrylate) (PDMAEMA). A hydrogel can be a temperature-sensitive hydrogel, an electrosensitive hydrogel, or even a light-sensitive hydrogel.

Aspect 19. The article of any one of Aspects 16-18, wherein the article is configured such that following application of about a 20% tensile strain to the article, the majority of the mechanically-activated microcapsules remain adhered to the matrix.

Aspect 20. The article of any one of Aspects 16-18, wherein the article is configured such that following application of about a 50% tensile strain to the article, the majority of the mechanically-activated microcapsules remain adhered to the matrix.

Aspect 21. The article of any one of Aspects 16-18, wherein the article is configured such that following application of about a 20% tensile strain to the article, the majority of the mechanically-activated microcapsules rupture.

Aspect 22. The article of any one of Aspects 16-18, wherein the article is configured such that following application of about a 50% tensile strain to the article, the majority of the mechanically-activated microcapsules rupture.

Aspect 23. The article of any one of Aspects 16-22, wherein the article is configured for exterior application to a subject.

Aspect 24. The article of any one of Aspects 16-22, wherein the article is configured for implantation into a subject.

Aspect 25. A method, comprising: treating a subject with an article according to any one of Aspects 16-24.

1. A composition, comprising:
a plurality of mechanically-activated microcapsules;
a mechanically-activated microcapsule defining a shell and an exterior surface; and
the mechanically-activated microcapsule comprising one or more adhesion groups disposed on the exterior surface of the mechanically-activated microcapsule,
the one or more adhesion groups being configured to effect a covalent interaction, a non-covalent interaction, or both between the one or more adhesion groups and a matrix material,
the covalent interaction, the non-covalent interaction, or both adhering the mechanically-activated microcapsule to the matrix material.

2. The composition of claim 1, wherein the one or more adhesion groups comprise a 1,2-dihydroxybenzene group.

3. The composition of claim 1, wherein the mechanically-activated microcapsule is characterized as biodegradable.

4. The composition of claim 1, wherein the mechanically-activated microcapsule comprises poly(D,L-lactide-co-glycolide).

5. The composition of claim 1, wherein the mechanically-activated microcapsule comprises a material enclosed within the shell of the mechanically-activated microcapsule.

6. The composition of claim 5, wherein the material comprises a therapeutic, an analgesic, anti-inflammatory, an antibiotic, or any combination thereof.

7. The composition of claim 6, wherein the material comprises an antibiotic.

8. The composition of claim 1, wherein the shell defines a thickness in the range of from about 0.05 μm to about 30 μm.

9. The composition of claim 1, wherein a mechanically-activated microcapsule defines a diameter of from about 0.5 μm to about 300 μm.

10. An injectable formulation, comprising:
a composition according to claim 1; and
a carrier,
the injectable formulation being configured for injection to a subject.

11. The injectable formulation of claim 10, wherein the matrix is a selected tissue of the subject.

12. The injectable formulation of claim 11, wherein the selected tissue is characterized as being in a disease state.

13. The injectable formulation of claim 12, wherein the disease state is a state of inflammation.

14. The injectable formulation of claim 11, wherein the one or more adhesion groups are configured to adhere preferentially to the selected tissue.

15. A method, comprising: injecting into a subject an injectable formulation according to claim 10.

16. An article, comprising:
a matrix material; and
a composition according to claim 1;
the composition adhered to the matrix material, and
the mechanically-activated microcapsules of the composition being adhered to the matrix by covalent interactions, non-covalent interactions, or both between the one or more adhesion groups of the mechanically-activated microcapsules and the matrix material.

17. The article of claim 16, wherein the matrix material is a fibrous material.

18. The article of claim 16, wherein the matrix material is a hydrogel.

19. The article of claim 16, wherein the article is configured such that following application of about a 20% tensile strain to the article, the majority of the mechanically-activated microcapsules remain adhered to the matrix.

20. The article of claim 16, wherein the article is configured such that following application of about a 50% tensile strain to the article, the majority of the mechanically-activated microcapsules remain adhered to the matrix.

21. The article of any claim 16, wherein the article is configured such that following application of about a 20% tensile strain to the article, the majority of the mechanically-activated microcapsules rupture.

22. The article of claim 16, wherein the article is configured such that following application of about a 50% tensile strain to the article, the majority of the mechanically-activated microcapsules rupture.

23. The article of claim 16, wherein the article is configured for exterior application to a subject.

24. The article of claim 16, wherein the article is configured for implantation into a subject.

25. A method, comprising: treating a subject with an article according to claim 16.
